(19) United States
(12) Patent Application Publication
(10) Pub. No.: US 2011/0190790 A1
(43) Pub. Date: Aug. 4, 2011

(54) Method For Operating A Medical Robot, Medical Robot, And Medical Work Place

(75) Inventors: Andreas Summerer, Ulm (DE); Thomas Neff, Munchen (DE); Tobias Ortmaier, Hemmingen (DE); Marc-Walter Ueberle, Friedberg (DE)

(73) Assignee: KUKA ROBOTER GMBH, Augsburg (DE)

(21) Appl. No.: 13/059,003

(22) PCT Filed: Aug. 7, 2009
(86) PCT No.: PCT/EP09/05753

§ 371 Date: Apr. 11, 2011

(30) Foreign Application Priority Data
Aug. 14, 2008 (DE) 10 2008 041 260.0

(51) Int. Cl. A61B 19/00

(52) U.S. Cl. 606/130

(57) ABSTRACT

The invention relates to a method for operating a telemanipulated medical robot (R) guided by hand or by means of an input device, to a telemanipulated medical robot (R) guided by hand or by means of an input device, and to a medical work place. The medical robot (R) comprises a robot arm (M) with a plurality of moveable axes (1-6) and a control device (17) for moving the axes (1-6) of the robot arms (M) by means of drives (11-16). The control device (17) is adapted to automatically change the work region (A) of the medical robot (R) due to a change of position, relative to a robot base (B) of the medical robot (R), of a living being (P) that is being treated by means of the medical robot (R) in such a way that the work region (A) of the medical robot (R) stays the same relative to the living being (P).

The invention relates to a method for operating a telemanipulated medical robot, in particular guided by hand or by means of an input device, a telemanipulated medical robot, in particular guided by hand or by means of an input device, and a medical work place.

Robots are working machines, which are equipped with tools for automatic handling and/or processing of objects, and are programmable in a plurality of motion axes, for example with regard to orientation, position and process sequence.

Robots are usable for example for medical and/or clinical applications, and are then for example part of a medical work place. For these applications, robots may also be guided remotely or directly.

US 2004/0077939 A1 for example discloses a medical work station with an X-ray apparatus, a surgical instrument, a position detecting system and a robot guiding the surgical instrument for treating a patient in an at least partially automated manner. In order to detect the positions of the surgical instrument, the X-ray apparatus and the patient, position markers are arranged on the X-ray apparatus, on the patient, and on the surgical instrument or on the robot, which are registered by an optical position detection apparatus of the position detection system. Based on an evaluation of the images of the position markers recorded with the optical position detection apparatus, it is possible to determine the location, i.e., the position and orientation of the position markers, and thus of the surgical instrument, of the X-ray apparatus and of the patient in the space.

In particular in the case of a surgical intervention, it is worth the effort so that if possible only tissue of the patient which is to be treated is treated with the robot or with the instrument guided by the robot.

The object of the invention is to create preconditions on the basis of which the danger of injury to a living being treated using a robot is at least reduced.

The object of the invention is fulfilled by a method for operating a telemanipulated medical robot, in particular guided by hand or by means of an input device, having the following procedural steps:
defining a work region of a medical robot intended for treating a living being, relative to the living being,
detecting a changing position or location of at least a part of the living being treated by means of the robot, and
automatically adjusting the work region of the medical robot on the basis of the changing position or location of the living being relative to the robot base, so that the work region of the medical robot remains the same relative to the living being.

The object of the invention is also fulfilled by a telemanipulated medical robot, in particular guided by hand or by means of an input device, having
a robot arm with a plurality of movable axes, and
a control device for moving the axes of the robot arm by means of drives, the control device being set up to automatically change the work region of the medical robot on the basis of a changing position or location of a living being treated by means of the medical robot relative to a robot base of the medical robot, so that the work region of the medical robot remains the same relative to the living being.

According to one embodiment of the robot or method according to the invention, the robot treating the living being is either telemanipulated or manually guided. The restriction of the work region can be represented to the attending physician through forces at the robot in the case of manually guided use, or through forces at an input station in the case of telemanipulated use. It is also possible that the robot in telemanipulated use does not allow itself to be moved beyond the work region.

It is thus possible to carry out the method according to the invention using the robot according to the invention.

The medical robot according to the invention is intended so that the living being, for example a human, is treated using it. To that end, a medical instrument, in particular a surgical instrument with which the living being is to be treated, is attached for example to an attaching device of the robot. The robot according to the invention may be programmed for example in such a way that it moves the medical instrument on a defined path. The robot according to the invention may also be guided remotely or directly, however. In order in particular to protect the living being during the treatment, the work region of the robot according to the invention is restricted. The work region of a robot is the permissible zone in which the robot may work and travel. When the robot according to the invention is in operation, in particular the so-called tool center point, and possibly also the axes of the robot, must be within a work region. That prevents the robot from encroaching on a prohibited region or leaving a predefined path.

As mentioned earlier, the robot according to the invention is used to treat the living being. As a rule, only a partial region of the body of the living being is treated, so that the work region of the robot according to the invention may be chosen so that the tool center point, and thus possibly the medical instrument moved by the robot according to the invention, is essentially only able to move within this partial region. That creates preconditions so that partial regions of the living being that are not to be treated are also not injured unintentionally by the robot according to the invention. According to the invention, the work region of the medical robot according to the invention is defined relative to the living being. This may be realized for example by defining the work region relative to a living being coordinate system assigned to the living being.

During the treatment of the living being, it is possible that the latter moves relative to the robot base, which is assigned for example to a robot coordinate system. The position or location which includes the position and orientation of the living being relative to the robot base, or relative to the robot coordinate system, changes accordingly. In order to do justice to such a change, according to the invention the work region of the medical robot according to the invention is adjusted on the basis of the changing position or location of the living being relative to the robot base or relative to the robot coordinate system. The work region of the medical robot according to the invention thus always remains the same relative to the living being.

The work region of the robot according to the invention may be delimited or set for example by means of a computer program running on the control device of the robot.

If the medical robot according to the invention is outside of its current work region, for example due to a movement of the living being relative to the robot base, then according to one embodiment of the method according to the invention or of the robot according to the invention, it is provided to move the medical robot automatically into its current work region. This is realized for example by setting up the control device of the robot according to the invention to move the robot arm automatically so as to guide the tool center point into the current work region, if the tool center point is located outside of the current work region due to the movement of the living being relative to the robot base.

According to one variant of the method according to the invention, the current position or location of the living being is detected by means of a navigation system. The control device of the robot according to the invention can accordingly be set up to ascertain the relative position or location of the living being relative to the robot base on the basis of the current position or location of the living being detected by means of the navigation system, in order to adjust the work region.

An additional aspect of the invention also relates to a medical work place, having the robot according to the invention and the navigation system communicating with the control device, which is set up to detect the current position or location of the living being, the control device being set up to ascertain the relative position or location of the living being relative to the robot base on the basis of the current position or location of the living being detected by means of the navigation system, in order to adjust the work region.

Navigation systems are generally known in medical technology, in particular in minimally invasive medical technology, for example from U.S. Pat. No. 6,895,268 B1. Navigation systems include a detection apparatus, which is for example an optical detection apparatus, which may for example have cameras, a laser tracking system, projectors for structured light or linear projectors. The detection apparatus is set up to detect markers or distinctive sites of the living being, for example on the living being, in particular on its surface, in a generally known way. On the basis of the markers or distinctive sites detected with the detection apparatus, a computing device of the navigation system is able to determine in an essentially generally known manner the position and possibly the orientation, i.e., the location of the living being.

Navigation systems are used, for example, during the operation to blend an instrument guided into the living being, for example the medical instrument moved by the robot according to the invention, into a picture of the living being taken prior to the operation. The picture of the living being is for example a 3-D image, which was taken for example with a computer tomograph or a magnetic resonance device. Blending the medical instrument into the picture taken before the operation generally requires a so-called registration of the image data record assigned to the preoperative picture to the interoperative situation, generally known to a person skilled in the art. With a rigid body situation, to this end for example a homogeneous coordinate transformation is determined, for example using corresponding points, which depicts both data records on each other.

According to another variant of the method according to the invention, the current location of the tool center point of the medical robot is ascertained by means of the navigation system and the current position or the current location of the living being relative to the robot base, based on the ascertained current locations of the living being and the tool center point. Accordingly, the control device of the robot according to the invention can be set up to ascertain, on the basis of the current location of the tool center point of the medical robot ascertained by means of the navigation system, the current position or current location of the living being relative to the robot base, based on the ascertained current locations of the living being and the tool center point, in order to adjust the work region.

In these variants of the robot according to the invention or of the medical work place according to the invention, the navigation system, or its computing device, communicates with the control device of the robot according to the invention. This can be realized for example in such a way that the navigation system and the control device of the robot according to the invention are connected with each other by means of a communication line or wirelessly, and communicate via a common communication protocol. It is possible via the latter to transmit status information, commands and/or data of the detection device of the navigation system.

An example of an exemplary embodiment of the invention is depicted in the attached schematic drawings. The figures show the following:

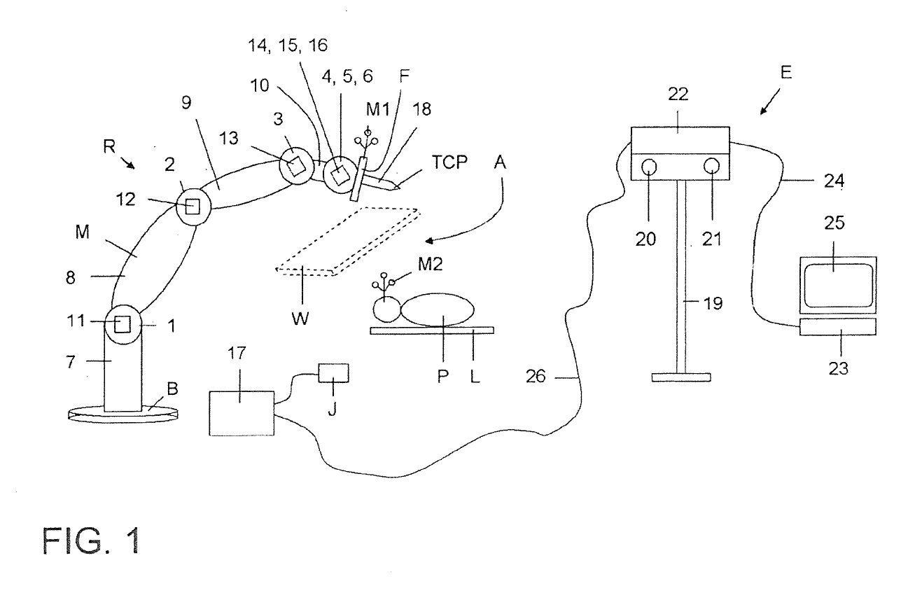

FIG. 1 a medical work place with a navigation system and a medical robot for treating a living being,

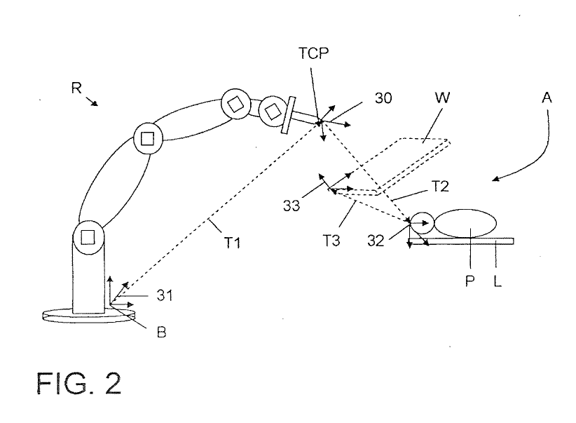

FIG. 2 coordinate systems assigned to the medical work place, and

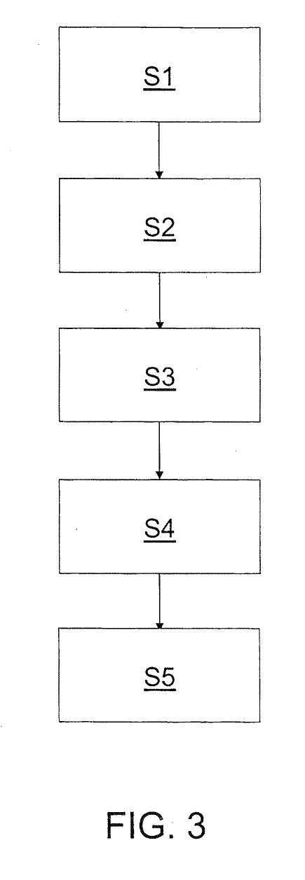

FIG. 3 a flow chart illustrating the operation of the medical robot.

FIG. 1 shows a medical workplace which has a medical robot R with a robot arm M. Robot arm M represents essentially the movable part of robot R, and includes a plurality of axes 1-6, a plurality of levers 7-10 and a flange F, to which in the case of the present exemplary embodiment a medical instrument 18 is attached.

Each of the axes 1-6 is moved with a drive, for example an electric drive 11-16, which are electrically connected in a non-depicted manner to a control computer 17 of robot R, so that control computer 17 or a computer program running on control computer 17 is able to activate electric drives 11-16 in such a way that the position of flange F of robot R, and thus surgical instrument 18 or its tool center point, can be oriented essentially freely in space. Electric drives 11-16 of robot R each include for example an electric motor and possibly power electronics that activate the motors.

In the case of the present exemplary embodiment, control computer 17 is designed in such a way that it, or a computer program running on it, is able to limit a work region A of robot R. The work region A of robot R is understood to mean the permissible zone for robot R for working and traveling. During operation of robot R, in the case of the present exemplary embodiment, surgical instrument 18 and in particular the tool center point TCP must be located within the work region A. In the case of the present exemplary embodiment, the work region A is bounded by a virtual wall W depicted with dashed lines in FIG. 1, the work region A of robot R being located beneath virtual wall W.

In the case of the present exemplary embodiment, robot R is intended to treat a patient P, lying on a patient-transport trolley L, with surgical instrument 18. In addition, in the case of the present exemplary embodiment, robot R is guided manually by a person not depicted, for example a doctor treating the patient, for example by pushing or pulling on robot arm M. Alternatively or additionally, robot R may also be moved by this person by telemanipulation, by means of an input device connected to control computer 17, for example a joystick J.

FIG. 1 also shows a navigation system that has a detection apparatus E which in the case of the present exemplary embodiment has two cameras 20, 21, markers M1 positioned on robot R, and markers M2 positioned on patient P. In the case of the present exemplary embodiment, detection apparatus E of the navigation system also includes a computer 22 and is attached to a stand 19, and the markers M1 of robot R are situated on its flange F.

Navigation systems as such are known to a person skilled in the art from sources including U.S. Pat. No. 6,895,268 B1, and are intended to determine the location, i.e., the position and orientation of an object, for example of patient P.

Navigation systems may be for example magnetic, or as in the case of the present exemplary embodiment, optical navigation systems, and are utilized for example to ascertain the position and possibly the orientation of an object. In order to ascertain for example the position of patient P or of the tool center point of robot R, the navigation system ascertains the positions of markers M1, M2 in space by means of its cameras 20, 21.

In the case of the present exemplary embodiment, computer 22 of detection device E is connected via an electric line 24 to a computer 23 having a monitor 25. An image data record assigned to a picture of patient P is stored in computer 23, the assigned picture of which may be displayed with monitor 25. The image data record was taken for example with an imaging medical device, for example a magnetic resonance device or a computer tomograph, prior to the treatment of patient P with robot R. During the treatment of patient P with robot R, an image of surgical instrument 18 may be blended into the image of patient P. To that end, the location of patient P and of surgical instrument 18 relative to the coordinate system of the image data record assigned to the image of the patient must be ascertained.

In the case of the present exemplary embodiment, computer 22 of detection device E is also connected to control device 17 of robot R by means of a communication line 26, so that computer 22 and control device 17 are able to communicate with each other in particular by means of a common communication protocol. Wireless communication between computer 22 and control device 17 is also conceivable.

FIG. 2 shows robot R, patient P situated on patient-transport trolley L, virtual wall W, and a TCP coordinate system 30 assigned to tool center point TCP of robot R, a robot coordinate system 31 assigned to robot R, whose origin falls on robot base B of robot R, a patient coordinate system assigned to patient P and a work region coordinate system 33 assigned to virtual wall W. In the case of the present exemplary embodiment, coordinate systems 30-33 are Cartesian coordinate systems.

As mentioned earlier, in the case of the present exemplary embodiment the tool center point TCP is supposed to be located only within work region A during treatment of patient P. During the treatment of patient P, work region A is also supposed to adjust to a potential movement of patient P relative to robot base B, hence in this case relative to robot coordinate system 31, caused for example by a movement of patient P or a movement of robot base B of robot R. This process is illustrated by means of a flow chart depicted in FIG. 3.

In the case of the present exemplary embodiment, work region A is first defined relative to patient P, in particular relative to patient coordinate system 32, step S1 of the flow chart of FIG. 3. In the case of the present exemplary embodiment, as already mentioned, work region A is bounded by virtual wall W, so that virtual wall W or its wall coordinate system 33 is defined relative to patient P or its patient coordinate system 32. This is described by a transformation T3. That results in pre-conditions such that surgical instrument 18 is not located outside of work region A during the treatment of patient P. Work region A may also be bounded for example by a plurality of walls or in some other way, for example by spheres, cylinders, cones or free-form surfaces.

During the treatment of patient P the detection system detects markers M1 and M2, whereby a computer program running on computer 22 is able to detect the location of patient P and hence the latter's patient coordinate system 32, and the location of flange F and hence the location of the tool center point TCP or its TCP coordinate system 30. That produces a transformation T2, which depicts the location of patient P from the perspective of the tool center point TCP, step S2 of the flow chart. This computer program can also run on the control device 17 of robot R, however. A registration was performed beforehand, as generally known to a person skilled in the art.

The location of the tool center point TCP or its TCP coordinate system 30 in the robot coordinate system 31 (coordinate system of robot base B) is depicted in the case of the present exemplary embodiment by a transformation T1, which is calculated for example from measurements of the angles of the joints of robot arm M, step S3 of the flow chart. Alternatively, it is also possible to affix another marker to the robot base B of robot R, whose position or location is detected by detection device E and is used to calculate the transformation T1.

The depiction of the virtual wall W, i.e., of the work region A in robot coordinate system 31, derives from the multiplication of the transformations T1, T2, T3 (Ti×T2×T3), step S4 of the flow chart.

Transformations T1 and T2 in the case of the present exemplary embodiment are updated for example at specified intervals, or when a specified change is exceeded, whereby a movement of patient P relative to the robot base B or relative to robot coordinate system 31 is recognized. That makes it possible for control device 17 to also update the work region A or the location of wall coordinate system 33, so that work region A always remains defined relative to patient P, step S5 of the flow chart.

Protection of risk structures while simultaneously carrying out the treatment of patient P by robot R on target structures is accordingly possible both when there is a movement of robot R relative to its robot base B and also when a virtual wall W is moved. The protection may be realized for the functional end of robot R, for example the surgical instrument 18, as well as for the structure of robot R.

If surgical instrument 18 is outside of the current work region A due to a movement of patient P relative to robot base B, then it is provided in the case of the present exemplary embodiment that control device 17 automatically moves surgical instrument 18 into the updated work region A.

If robot R is guided manually, then in the case of the present exemplary embodiment robot R can no longer be moved further manually, or only with the application of increased manual force, when robot R leaves work region A. This can be achieved for example by having control computer 17 activate drives 11-16, so that these exert a torque on levers 7-10.

If robot R is moved by telemanipulation using joystick J, then in the case of the present exemplary embodiment robot R can no longer be moved further, or joystick J generates a tactile feed-back to the person when robot R leaves work region A.

1. A method for operating a medical robot, in particular guided manually or telemanipulated by means of an input device, having the following procedural steps:
defining a work region (A) of a medical robot (R) provided for treating a living being (P), relative to the living being,
detecting a changing position or location of at least a part of the living being (P) to be treated by means of the medical robot (R), relative to the robot base (B) of the medical robot (R), and
automatically adjusting the work region (A) of the medical robot (R) on the basis of the changing position or location of the living being (P) relative to the robot base (B), so that the work region (A) of the medical robot (R) remains the same relative to the living being (P).

2. The method according to claim 1, having automatic movement of the medical robot (R) into its current work region (A) when the medical robot (R) is located outside of its current work region (A) due to a movement of the living being (P) relative to the robot base.

3. The method according to claim 1 or 2, having detection of the current position or location of the living being (P) by means of a navigation system (E, M2).
